(19) United States
(12) Patent Application Publication
(10) Pub. No.: US 2025/0166829 A1
(43) Pub. Date: May 22, 2025

(54) AUTOMATED DISEASE DETECTION USING RETINAL IMAGES

(71) Applicant: Welch Allyn, Inc., Skaneateles Falls, NY (US)

(72) Inventors: David E. Quinn, Auburn, NY (US); Autumn Thompson, Philadelphia, PA (US)

(21) Appl. No.: 18/953,876

(22) Filed: Nov. 20, 2024

Related U.S. Application Data
(60) Provisional application No. 63/601,463, filed on Nov. 21, 2023.

(51) Int. Cl. G16H 50/20; G06T 7/00; G16H 10/60; G16H 30/40; G16H 50/30

(52) U.S. Cl. CPC G16H 50/20; G06T 7/0012; G16H 10/60; G16H 30/40; G16H 50/30; G06T 2207/20081; G06T 2207/30041; G06T 2207/30101

(57) ABSTRACT

A patient screening system for providing recommendations for screening of potential diseases or disease risk(s) of a patient based on their health records and retinal images, is described herein. The patient screening system may include an optical imaging device operable at a doctor's office, and associated methods configured to generate the recommendation. The patient screening system may implement various AI/ML models trained on a training dataset of anonymized patient data. The patient screening system may be based on discovering correlations between features of the retinal images and the health records in the training dataset, and corresponding disease diagnoses included in the health records. The patient screening system may also implement classifiers for various diseases based on data in the training dataset. Any patient screening based on the recommendation may be followed up, and results of such screening used to improve performance of the patient screening system.

### RELATED APPLICATIONS

This Patent Application is a nonprovisional of and claims priority to U.S. Provisional Patent Application No. 63/601,463, entitled “AUTOMATED DISEASE DETECTION USING RETINAL IMAGES,” filed on Nov. 21, 2023, the entirety of which is incorporated herein by reference.

### TECHNICAL FIELD

This application relates to techniques for automatically detecting potential diseases based on retinal images and patient health records, and providing recommendations for further screening related to the detected diseases.

### BACKGROUND

Vision screening, such as retinal scans, typically includes screening for diseases of the eye. However, many systemic diseases manifest detectable signs in retinal scans of a patient, sometimes even in early or otherwise asymptomatic phase of a disease. Retinal scans are non-invasive and can be easily administered in a primary care doctor's office as a part of regular health screening. However, a primary care doctor may not be able to review the retinal scans for signs of disease. A manual analysis of retinal scans of a patient by a retina specialist, in addition to adding to a cost and complexity of a health screening, may also fail to flag early signs of a disease because the retina specialist may not be familiar with a patient's overall medical history.

Accordingly, it would be advantageous to be able to screen a patient for a host of potential diseases automatically. Examples of diseases that may be screened for may include heart diseases, kidney diseases, neurodegenerative diseases, anemia, sleep apnea, fibromyalgia, multiple sclerosis, and the like.

The various examples of the present disclosure are directed toward overcoming one or more of the deficiencies noted above.

### SUMMARY

In an example of the present disclosure, a method includes receiving an image of a retina of an eye of a patient, receiving, by the processor and from an electronic medical record (EMR) of the patient, patient data corresponding to the patient, determining a feature in the image, and determining, by inputting the feature and at least a portion of the patient data as input to a machine learning (ML) model, a confidence level associated with a first disease. The method also includes determining, based on the confidence level being higher than a threshold, a recommendation for screening of the patient based on the first disease, and providing, by a processor and to an output device, an output indicating the recommendation.

In another example of the present disclosure, a system includes memory, a processor and computer-executable instructions stored in the memory and executable by the processor. The instructions, when executed, cause the processor to perform operations comprising: receiving an image of a retina of an eye of a patient, receiving, from an electronic medical record (EMR) of the patient, patient data corresponding to the patient, determining a feature in the image, and determining, by inputting the feature and at least a portion of the patient data as input to a machine learning (ML) model, a confidence level associated with a first disease, determining, based on the confidence level being higher than a threshold, a recommendation for screening of the patient based on the first disease, and providing, to the EMR of the patient, an output indicating the recommendation.

In still another example of the present disclosure, a non-transitory computer-readable storage medium storing processor-executable instructions that, when executed, cause one or more processors to: receive, from an optical imaging device, an image of a retina of an eye of a patient, access, from an electronic medical record (EMR) storage, EMR data of the patient, determine a feature in the image, determine, by inputting the feature and at least a portion of the EMR data as input to a machine learning (ML) model, a confidence level associated with a first disease, and determine, based on the confidence level being higher than a threshold, a recommendation for screening of the patient based on the first disease.

### BRIEF DESCRIPTION OF THE DRAWINGS

Features of the present disclosure, its nature, and various advantages, may be more apparent upon consideration of the following detailed description, taken in conjunction with the accompanying drawings.

FIG. 1 illustrates an example environment for recommending screening for one or more diseases that may be identified from a combination of retinal scan(s) and health records of a patient.

FIG. 2 illustrates a first block diagram of an example process for training a recommendation system for identifying diseases or disease risk, as described herein.

FIG. 3 illustrates a second block diagram of an example process for generating a recommendation for screening for one or more diseases, as described herein.

FIG. 4 provides a first flow diagram illustrating an example method of the present disclosure.

FIG. 5 provides a second flow diagram illustrating an example method of the present disclosure.

FIG. 6 illustrates at least one example device configured to enable and/or perform the some or all of the functionality discussed herein.

In the figures, the left-most digit(s) of a reference number identifies the figure in which the reference number first appears. The use of the same reference numbers in different figures indicates similar or identical items or features. The drawings are not to scale.

### DETAILED DESCRIPTION

The present disclosure is directed, in part, to a disease identification system programmed or otherwise configured to generate a recommendation for further screening, and corresponding methods. Such an example disease identification system may be configured to accept, as inputs, one or more retinal images of a patient, and the patient's electronic health records, and generate, as output, the recommendation for further screening. Although many retinal findings are sometimes non-exclusive to a particular disease, when viewed in combination with medical information in a patient's health records, such findings may indicate early signs of specific disease(s), and may enable a vision screening system to recommend further screening for the disease(s). Such screening may help prevent progression of the disease(s) to serious levels and/or prevent life threatening conditions by allowing early detection and treatment of the disease(s).

In examples, the disease identification system may leverage artificial intelligence (AI)-generated correlations between features of retinal scans and information in the patient's medical history, including test results and trends. Such a disease identification system may be included in a patient health screening, where an operator may capture the retinal images using a vision screening device and the recommendation may be made available to the operator or to a clinician, who may be different from the operator. The disease identification system may determine, based on analysis of the retinal images and information in the health records, one or more diseases correlated with features of the retinal images and/or information in the health records. Specifically, the present disclosure is directed to methods for screening for systemic diseases and/or ophthalmic diseases that develop over time and would otherwise require complex and/or invasive testing to diagnose. In some examples, such diseases may require inputs from multiple specialist doctors, and may therefore, not be diagnosed at an early stage. The methods of the present disclosure may recommend screening for such diseases based on data available during a visit to a medical appointment at a primary care doctor's office.

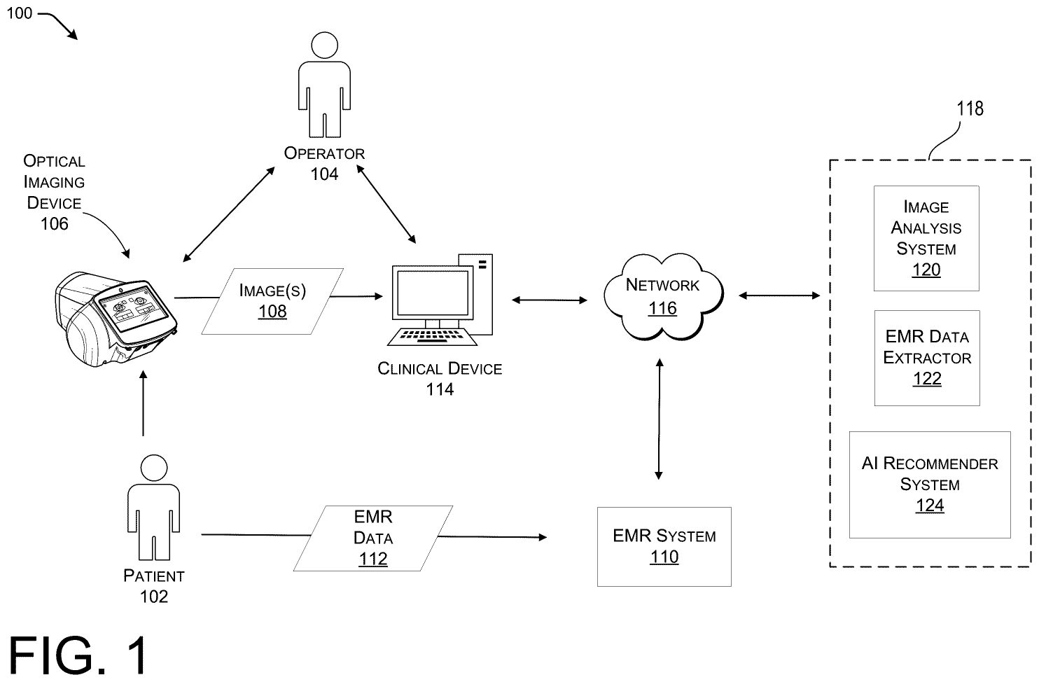

Based at least in part on a confidence level associated with the determination of the one or more diseases, the system may generate an output including at least one of a recommendation or a diagnosis associated with the patient. Such an output (e.g., the recommendation and/or the diagnosis) may be indicative of the disease(s) and/or disease risk(s) detected, indicate that the patient requires additional screening, and/or indicate that the screening was normal (e.g., did not detect any disease with a confidence level that exceeded a threshold level). In examples, the system may determine the disease(s) by using trained machine learning (ML) model(s) or other AI techniques which have been trained on anonymized data from large numbers of patient health records, including disease diagnoses and medical test results over an extended period of time, and corresponding retinal images. In some examples, the AI techniques may include data-driven discovery of correlations between data associated with patients and disease diagnoses. In such examples, the recommendation may be based on the discovered correlations, including correlations of diseases with trends in the data. Various implementations of the present disclosure will be described in detail with reference to FIGS. 1-6. It is to be appreciated that while these figures describe methods and systems of the present disclosure, any examples set forth in this specification are not intended to be limiting and merely set forth some of the many possible implementations.

FIG. 1 illustrates an example environment 100 for screening a patient 102 to identify disease(s) and/or disease risk(s) that may be exhibited by the patient 102. The screening may be based at least in part on imaging an eye of the patient 102 by an operator 104 using an optical imaging device 106. The patient 102 may be any individual being monitored in a clinical environment e.g., a person presenting for a medical appointment at a doctor's office. In examples, the patient 102 may be screened at a primary care doctor's office during a periodic health checkup, and the operator 104 may be a healthcare provider, such as a physician, a physician's assistant (PA), a nurse, a medical student, a nursing student, a medical technician, and the like.

In examples, the optical imaging device 106 may be configured to obtain one or more images 108 of a retina and/or a fundus (which includes a back surface of an eye comprising the retina, macula, optic disc, fovea, and blood vessels) of at least one eye of the patient 102. In various implementations, the optical imaging device 106 may include an optical coherence tomography (OCT) camera configured to obtain OCT or OCT angiography (OCTA) images of the eye(s) of the patient 102. In some cases, the optical imaging device 106 may comprise a slit lamp imaging device configured to obtain slit lamp images (or projection images) of the eye(s) of the patient 102. In some examples, the optical imaging device 106 may include at least one fluorescence camera configured to obtain one or more fluorescence angiograms of the eye(s) of the patient 102. In some examples, the optical imaging device 106 may be configured to generate one or more color fundus (e.g., retinal) photography (CFP) images of the eye(s) of the patient 102, one or more fluorescein angiography (FA) images of the eye(s) of the patient 102, one or more indocyanine green (ICG) angiography images of the eye(s) of the patient 102, one or more fundus autofluorescence (FAF) images of the patient, or any combination thereof.

The environment 100 may also include an electronic medical record (EMR) system 110 configured to store EMR data 112 associated with the patient 102. As used herein, the terms “electronic health record,” “electronic medical record,” “EMR,” and their equivalents, may broadly refer to stored data, in any modality of storage (e.g., temporary, transitory, permanent, etc.), indicative of a medical history and/or medical condition(s) of an individual, wherein the stored data is accessible (e.g., can be modified and/or retrieved) by one or more computing devices. An EMR data of an individual may include data indicating previous or current medical diagnoses, diagnostic tests, or treatments of the individual. In addition, the EMR data may indicate demographics of the individual (e.g., age, sex, race, etc.), parameters (e.g., vital signs, blood pressure, body mass index (BMI), etc.) of the individual, lifestyle information (e.g., smoking or drug use status, physical activity level, diet, alcohol use, etc.), notes from one or more medical appointments attended by the individual, medications prescribed or administered to the individual, therapies (e.g., surgeries, outpatient procedures, etc.) administered to the individual, results of diagnostic tests performed on the individual, identifying information (e.g., a name, birthdate, etc.) of the individual, or a combination thereof. In some examples, the EMR system 110 may be implemented on one or more servers, such as servers located at a data center.

In some examples, the EMR system 110 may be connected to a clinical device 114 via a network 116. The clinical device 114 can include a computing device, such as a device including at least one processor configured to perform operations. In some cases, the operations are stored in memory in an executable format. Examples of computing devices include a personal computer, a tablet computer, a smart television (TV), a mobile device, a mobile phone, or an Internet of Things (IoT) device. In some examples, the clinical device 114 may be operated by the operator 104, and may receive the image(s) 108 captured by the optical imaging device 106. The clinical device 114 may provide a user interface to the operator 104 e.g., to access or enter data related to the patient 102 and/or view the image(s) 108 captured by the optical imaging device 106. In examples, the clinical device 114 or the optical imaging device 106 may store the image(s) 108 of the eye(s) of the patient 102 in the EMR system 110 in association with the EMR data 112 of the patient 102.

In examples, the network 116 may represent one or more communication networks. Examples of communication networks include at least one wired interface (e.g., an ethernet interface, an optical cable interface, etc.) and/or at least one wireless interface (e.g., a BLUETOOTH interface, a WI-FI interface, a near-field communication (NFC) interface, a Long-Term Evolution (LTE) interface, a New Radio (NR) interface, etc.). In some examples, data or other signals may be transmitted between elements of FIG. 1 over a wide area network (WAN), such as the Internet. In some cases, the data may include one or more data packets (e.g., Internet Protocol (IP) data packets), datagrams, or a combination thereof.

In various examples, the clinical device 114 may be connected, via the network 116, to a remote computing device 118, such as a server implemented on a cloud platform. In examples, the clinical device 114 may upload, to the remote computing device 118, the image(s) 108 captured by the optical imaging device 106. The remote computing device 118 may implement an image analysis system 120 that receives, as an input, the image(s) 108 captured by the optical imaging device 106 and analyze the image(s) 108 to determine various features. In other examples, the optical imaging device 106 may be in direct communication with the remote computing device 118 to upload the image(s) 108, and/or the image analysis system 120 may be implemented, completely or in part, on the clinical device 114.

In some examples, the image analysis system 120 may implement one or more image processing components to identify significant landmark(s) in the image(s) 108 of the eye(s). As used herein, the term “landmark,” and its equivalents, may refer to an anatomical structure that is observed in healthy or diseased eyes. Examples of landmarks include one or more of a macula, an optic disc (OD), a retina, a cornea, an iris, a lens, one or more retinal layers, one or more blood vessels, or a fovea. The one or more image processing components may comprise first machine learning (ML) models configured to output a location of one or more of a set of landmarks in an input image of the back of the eye(s). For example, the location may be indicated by identifying pixels in the input image that correspond to an area of the respective landmark.

In some examples, the image analysis system 120 may implement feature detectors to identify different types of ophthalmic features in the image(s) 108. As used herein, the term “feature,” and its equivalents, may refer to a structure or visible sign within an image of an eye that may be correlated with one or more diseases and/or medical conditions. Examples of ophthalmic features may include one or more of a microaneurysm, a hemorrhage, drusen, exudate, edema, a cup/disc ratio (CDR), focal arteriolar narrowing, arterio-venous nicking, a cotton wool spot, an embolus, a red spot, retinal whitening, a Hollenhorst plaque, a Roth spot, a microinfarct, coagulated fibrin, new vessels elsewhere (NVE), a vitreous hemorrhage (VH), a pre-retinal hemorrhage (PRH), new vessels on a disc (NVD), venous beading, an intraretinal microvascular abnormality (IRMA), diameter and topology of blood vessels, retinal vascular caliber, average diameter of retinal arterioles and venules summarized as arteriovenous ratio (AVR), etc.

In some examples, one or more of the feature detectors may comprise second machine learning (ML) models configured to output a binary indication of whether the respective feature is present or not along with a confidence score, and/or a location of the feature in an input image. Additionally, the image analysis system 120 may output various characteristics of the detected ophthalmic features, such as the location of the feature in the image (e.g., a quadrant of the eye the feature is located, a distance and/or direction from one or more of the landmarks, a proximity to the one or more landmarks, and the like), and a size of the feature in the image(s) (e.g., relative to size(s) of the landmark(s), as a number of pixels, as a fraction of area of the feature of the total retinal area, etc.). In examples, the image analysis system 120 may also implement image processing techniques to determine one or more measurements associated with the detected ophthalmic features e.g., diameter of a blood vessel, number of spots, density of spots, textural elements of the feature, etc.

In some examples, the image analysis system 120 may also determine ophthalmic features indicating color(s) associated with the landmarks in the eye. For example, the image analysis system 120 may determine ophthalmic features indicating an average color value (e.g., in a color space such as RGB, HSI, CIE L*a*b*, CIE L*u*v*, etc.) of the optic disc, the blood vessels, the fovea, or the retina. The image analysis system 120 may also determine ophthalmic features indicating relative intensities between various landmarks or areas of the fundus.

Additionally, in some examples of the present disclosure, the image analysis system 120 may generate a standardized retinal image from the image(s) 108 captured by the optical imaging device 106. In examples, the image analysis system 120 may generate the standardized retinal image by scaling (e.g., to a standard size and aspect ratio) and normalizing (e.g., histogram stretching to cover full range of brightness and contrast levels) the image(s) captured by the optical imaging device 106, such that the landmarks of the retina (e.g., optical disc, fovea, important blood vessels, etc.) are located at pre-determined positions relative to an image boundary of the standardized retinal image. For example, after such standardization, the landmarks may be in alignment in multiple different standardized retinal images (e.g., occurring at a same location relative to the respective image boundaries).

In examples, the image analysis system 120 may associate a timestamp indicating a date/time of capture of the image(s) 108 with each of the ophthalmic features and the standardized retinal image determined from the image(s) 108.

In examples, the first ML models and the second ML models of the image analysis system 120 may be pre-trained based on training images e.g., from a training dataset. For example, the first ML models may be trained on a first training dataset including images of the back of the eye(s) (e.g., retinal images) labeled with the set of landmarks, and the second ML models may be trained on a second training dataset, the second training dataset including example images depicting the ophthalmic features, as well as ground truth information associated with each image identifying the ophthalmic feature depicted therein. In examples, one or more expert annotators may review the images in the training datasets and indicate, as ground truth information, the landmarks and/or whether the respective images depict one or more of the ophthalmic features.

In some examples, the image analysis system 120 may also be configured to confirm an image quality of the image(s) 108 captured by the optical imaging device 106. As used herein, the term “image quality,” and its equivalents, may refer to an extent to which an image accurately represents a subject or other item depicted in the image. Several factors may be associated with image quality, such as a blurriness of the image or other distortions in the image. In some examples, if a quality of the image(s) is determined to be below a threshold, the image analysis system 120 may refrain from analyzing the image(s) and/or generate a notification indicating that the image(s) are of an insufficient quality. The image analysis system 120 may transmit the notification to the clinical device 114, and based on the notification, the operator 104 may retake the image(s) using the optical imaging device 106. In examples in which the medical image analysis system 120 confirms that the image(s) are of a sufficient quality, the image analysis system 120 may perform further analysis on the image(s) to detect the ophthalmic features. Techniques for identifying example diseases based on the ophthalmic features of the image(s) 108 (e.g., as determined by the image analysis system 120) are described in U.S. patent application Ser. No. 17/709,950, filed Mar. 31, 2022, titled “Automated disease identification based on ophthalmic images,” which is hereby incorporated by reference in its entirety and for all purposes.

In examples, the remote computing device 118 may also implement an EMR data extractor component 122. The EMR data extractor component 122 may process the EMR data 112 to extract data of interest, which may include demographic data (e.g., age, sex, race, etc.), health parameters (e.g., vital signs, blood pressure, body mass index (BMI), etc.), lifestyle information (e.g., smoking or drug use status, physical activity level, diet, alcohol use, etc.), geographic region of residence, previous or current medical diagnoses, diagnostic tests and results, medical treatments, prescriptions, etc. The EMR data extractor component 122 may also ignore some data of the EMR data 112 (e.g., a name, address, emergency contact, etc.) based on irrelevance to disease status. In examples, the EMR data extractor component 122 may convert the data of interest from the EMR data 112 to a pre-defined standard representation. As an example, the EMR data extractor component 122 may create a vector (e.g., an EMR feature vector), which may be an one or two-dimensional array with data fields indicating the data of interest from the EMR data 112, and associate a timestamp with each data field indicating a date/time when the data of interest was determined or added to the EMR data 112. In some examples, the EMR data extractor component 122 may include, in the EMR feature vector, a first set of data (e.g., numerical values) from the EMR data 112 in their respective native form (e.g., age, BMI number, blood glucose level, etc.), represent a second set of data as levels (e.g., 1-3, 1-5, 1-10, etc.) corresponding to ranges of values (e.g., 1: low, 2: medium, 3: high), and represent a third set of data (e.g., textual data) as category numbers (e.g., 0: non-smoker, 1: smoker in a “smoking status” data field, 0: sedentary, 1: low activity, 2: medium activity, 3: active in an “activity level” data field, and so on). In some examples, the EMR data extractor component 122 may also divide numerical values of the EMR data 112 into range levels and enter the respective range level in the EMR feature vector. For example, an age of a patient may be divided into 1: under 2, 2: 2-5 years, 3: 5-11 years, 4: 12-18 years, 5: 18-54 years, and 6: 55+ years, where the EMR feature vector includes a range level indicator (1-6) corresponding to the age of the patient 102. As another example, an address of a patient (e.g., postal code, city name, etc.) may be mapped to a broader geographic region (e.g., county, state, portion of a country, etc.) and included in the EMR feature vector.

In examples, the remote computing device 118 may also implement an AI (e.g., artificial intelligence-based) recommender system 124 to generate recommendations for the patient 102 based on the image(s) 108 and the EMR data 112. For example, the image analysis system 120 may provide the detected ophthalmic features and/or the standardized retinal image, as inputs, to the AI recommender system 124, and the EMR data extractor 122 may provide the representation of the EMR data 112, as inputs, to the AI recommender system 124.

In examples, the AI recommender system 124 may be configured to determine whether the patient 102 is exhibiting one or more diseases or disease risks, and based on the determination, generate a recommendation. As used herein, the term “disease,” and its equivalents, may refer to a pathology or a health risk. Examples of diseases that may be identified by the AI recommender system 124 include at least one of heart disease, kidney disease, anemia, Alzheimer's, Parkinson's, multiple sclerosis, obstructive sleep apnea, fibromyalgia, Lyme disease, stroke risk, heart disease risk, kidney disease risk, and the like. In examples, the AI recommender system 124 may also receive, from the clinical device 114 and/or directly from the EMR system 110, the EMR data 112 associated with the patient 102. In various examples, the AI recommender system 124 may use, as inputs, the image(s) 108 captured by the optical imaging device 106, the ophthalmic features and/or standardized images determined by the image analysis system 120, the EMR data 112 associated with the patient 102, or a combination thereof, to determine the one or more diseases or disease risk(s) exhibited by the patient 102.

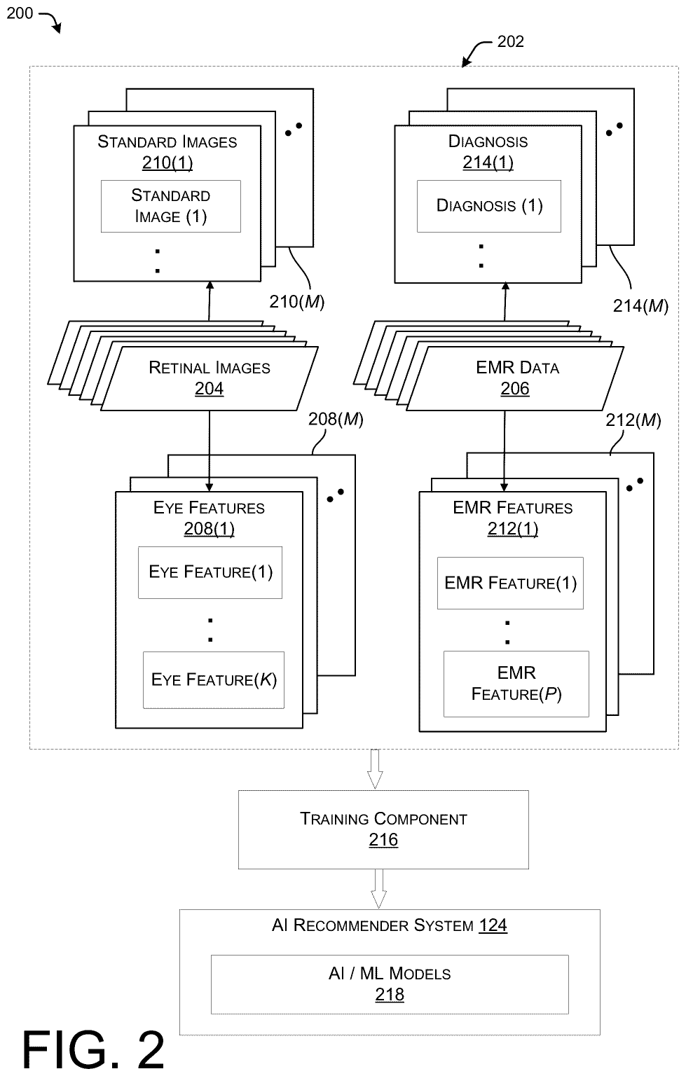

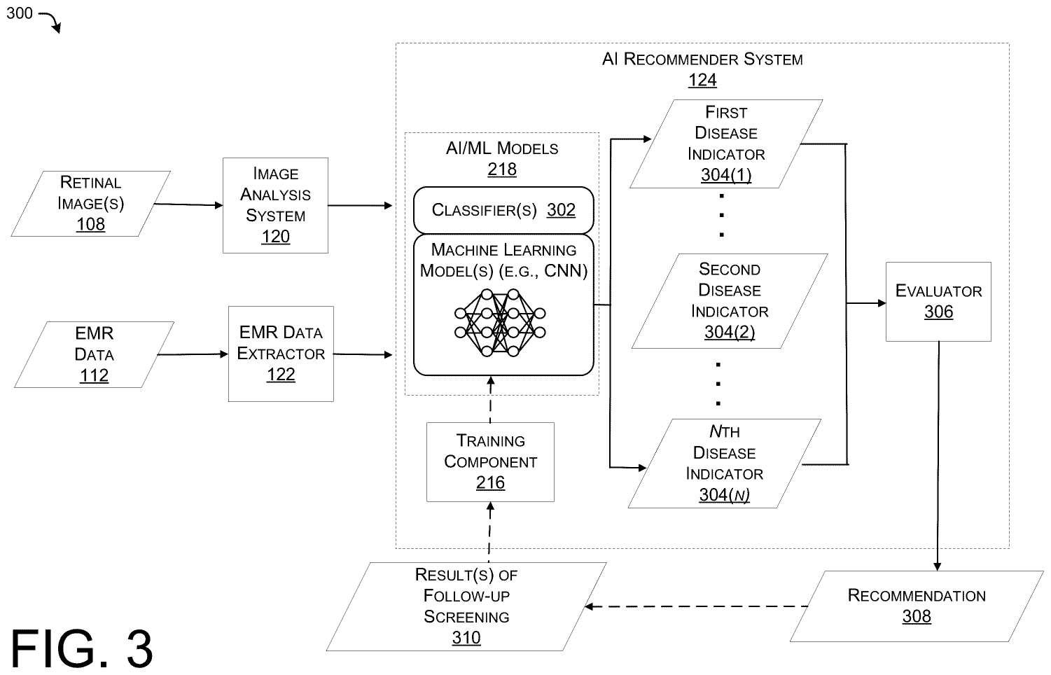

In examples of the present disclosure, the AI recommender system 124 may comprise machine learning models, expert systems, statistical models, and the like, trained on large sets of anonymized training data comprising patient health records, including disease diagnoses and medical test results over an extended period of time, and corresponding retinal images. Such training data may be accumulated from patient data in the EMR system 110 and/or data from publicly available health studies e.g., heart disease risk studies, sleep apnea studies, diabetes risk studies, etc. In some examples, during a training phase, the AI recommender system 124 may implement data mining techniques on the large sets of anonymized training data to discover correlations between features in the data and disease diagnoses. For example, the AI recommender system 124 may determine a correlation between a feature set including optic disc edema, high blood pressure, and high BMI, and a diagnosis of obstructive sleep apnea. Based on this correlation, the AI recommender system 124 may generate a recommendation for screening for sleep apnea when the patient 102 presents with the matching feature set. The AI recommender system 124 and the training phase of the AI recommender system 124 are described in further detail with reference to FIGS. 2 and 3.

In examples, the AI recommender system 124 may generate a recommendation based on the one or more diseases and/or disease risk(s) determined to be exhibited the patient 102. For example, the recommendation may indicate disease(s) or disease risk(s) detected, indicate that the patient requires additional screening, or indicate that the screening was normal (e.g., no disease(s) detected). In some examples, the recommendation may also include an associated urgency level e.g., follow-up with screening in 6-12 months, follow-up with screening within 3 months, immediate attention needed, etc., based on a severity of the disease(s) or disease risk(s). In some examples, the AI recommender system 124 may transmit the recommendation to the clinical device 114, where the recommendation may be output, via a user interface, to the operator 104. Accordingly, the operator 104 may take various actions to provide the recommendation to the patient 102, schedule follow-up screening, and/or add the recommendation to the EMR data associated with the patient 102. In some examples, the AI recommender system 124 may transmit the recommendation to the EMR system 110. In such examples, the recommendation may be accessed from the EMR system 110, at a later time, by other users (e.g., physicians, nurses, etc.) caring for the patient 102.

In some examples, there may be different standards of practice for recommendations when a particular disease is suspected, based on a country or geographic region. For example, the recommended follow-up screenings and/or treatments for the particular disease may be different in the United States from the United Kingdom. In some examples, the variations in the standards of practice may be based on a prevalence of the particular disease in the geographic location and/or guidelines of a health authority in the geographic region. In various implementations, the AI recommender system 124 may determine the recommendation according to standards of practice in a geographic location of the environment 100. In some examples, the AI recommender system 124 may also take into account guidelines from an insurance policy of the patient 102 (e.g., as indicated in the EMR data) to determine the recommendation.

As used herein, the terms “machine learning,” “ML,” and their equivalents, as used with reference to the image analysis system 120 and the AI recommender system 124, may refer to a computing model that can be optimized to accurately recreate certain outputs based on certain inputs. In some examples, the ML models include deep learning models, such as convolutional neural networks (CNN), recurrent neural networks (RNN), transformers, any combination thereof, or other types of NNs. The term Neural Network (NN), and its equivalents, may refer to a model with multiple hidden layers, wherein the model receives an input (e.g., at least one vector, matrix, or tensor) and transforms the input by performing operations via the hidden layers. An individual hidden layer may include multiple “neurons,” each of which may be disconnected from other neurons in the layer. An individual neuron within a particular layer may be connected to multiple (e.g., all) of the neurons in the previous layer, based on the model architecture. In some examples, a NN may further include at least one fully-connected layer that receives a feature map output by the hidden layers and transforms the feature map into the output of the NN. The output of an NN can be in any form based on the purpose of the learning network. For example, the output can be a name of a detected feature, a location of the detected feature, an indication of the presence of the detected feature, or any combination thereof.

As used herein, the term “CNN,” and its equivalents and variants, may refer to a type of NN model that performs at least one convolution (or cross correlation) operation on an input image and may generate an output image based on the convolved (or cross-correlated) input image. A CNN may include multiple layers that transforms an input image (e.g., an ophthalmic image) into an output image via a convolutional or cross-correlative model defined according to one or more parameters. The parameters of a given layer may correspond to one or more filters, which may be digital image filters that can be represented as images (e.g., 2D images). A filter in a layer may correspond to a neuron in the layer. A layer in the CNN may convolve or cross correlate its corresponding filter(s) with the input image in order to generate the output image. In various examples, a neuron in a layer of the CNN may be connected to a subset of neurons in a previous layer of the CNN, such that the neuron may receive an input from the subset of neurons in the previous layer, and may output at least a portion of an output image by performing an operation (e.g., a dot product, convolution, cross-correlation, or the like) on the input from the subset of neurons in the previous layer. The subset of neurons in the previous layer may be defined according to a “receptive field” of the neuron, which may also correspond to the filter size of the neuron. Other types of NN frameworks can also be used. For example, the image analysis system 120 may include one or more transformer-based models. For instance, a transformer-based model can be used as backbones for NNs of the image analysis system 120 and/or the AI recommender system 124. For example, the transformer-based model may comprise an encoder component generating embeddings by mapping input(s) to a high-dimensional embedding space, and such embeddings may be used as features, alternatively or in addition to the features detected by the image analysis system 120. In some examples, the embeddings generated by the encoder component may be used as input features for other ML models used by the image analysis system 120 and/or the AI recommender system 124.

It should be understood that, while FIG. 1 depicts a single optical imaging device 106, in additional examples, the environment 100 may include any number of local or remote optical imaging devices configured to operate independently and/or in combination to capture various types of retinal images. Additionally, although FIG. 1 illustrates the optical imaging device 106, the EMR system 110, the clinical device 114, and the remote computing device 118 as separate entities, in some implementations, one or more of these entities may be correspond to the same computing device.

As discussed herein, FIG. 1 depicts an exemplary environment 100 that includes components for capturing retinal images of a patient, and based on the retinal images and additional information from health records of the patient, providing, by a trained AI-based recommender system, recommendation(s) for screening the patient for disease(s) and/or disease risk(s).

FIG. 2 illustrates an example system 200 for training the AI recommender system 124 based on a large, anonymized training dataset 202. The AI recommender system 124 may be trained to discover correlations between patient data (e.g., retinal images and EMR data) and disease diagnoses or risk of future disease(s), and/or train classifiers for detecting one or more diseases or disease risk(s) based on the patient data. In some examples, the training dataset 202 may be stored on a data server (e.g., cloud memory storage) different from the remote computing device 118.

As illustrated in FIG. 2, the training dataset 202 may comprise retinal images 204 and EMR data 206 of M different individual patients, each patient corresponding to a data instance, m, where m=1, . . . , M. In examples, the image analysis system 120, as described with reference to FIG. 1, may process the retinal images 204 to extract eye features 208(1)-208(M) and/or generate one or more standard images 210(1)-210(M), each corresponding to a data instance (e.g., a particular patient's data). In an example shown in FIG. 2, each of the eye features 208 comprise K features (1, . . . , K). In some examples, the K features may include multiple instances of a same ophthalmic feature (e.g., AVR ratio, optic disc edema, etc.) associated with different timestamps (as indicated by the image analysis system 120 based on the date and/or time of day when the underlying retinal images 204 captured), providing historical data of the eye features 208 for each patient. Examples of the eye features 208 are described in U.S. patent application Ser. No. 17/709,950, filed Mar. 31, 2022, titled “Automated disease identification based on ophthalmic images,” which is incorporated by reference herein, as noted above.

Similarly, the EMR data extractor 122, as described with reference to FIG. 1, may process the EMR data 206 to extract EMR features 212(1)-212(M), each corresponding to a data instance, m, of the M data instances. For example, the EMR data extractor 122 may issue a query to the EMR system 110 (e.g., to a database storing EMR information) requesting anonymized data, and may receive, in response, the EMR data 206, and associations with corresponding data of a same patient in the retinal images 204. In another example, the EMR data extractor 122 may be provided a read-only access to the EMR system 110 to extract the EMR data 206, without accessing the patient's identifying information. In the example shown in FIG. 2, each of the EMR features 212 comprise P features (1, . . . , P). The EMR data extractor 122 may also extract one or more diagnosis 214(1)-214(M), each corresponding to a data instance. For example, some data instances may contain multiple diagnoses in the corresponding EMR data, whereas, other data instances may have no diagnosis in the EMR data. In some examples, the EMR data extractor 122 may enter a diagnosis of “normal health” when the corresponding EMR data does not include any disease diagnosis. In some examples, each of the P EMR features and the diagnosis 214 may be associated with a timestamp indicating a date and/or time of day of collection of the underlying EMR data. For example, “diagnosis(1)” of the diagnosis 214(1) may indicate “normal” at a first time, and a “diagnosis(2)” of the diagnosis 214(1) may indicate “stroke” at a second time after the first time, for the same data instance (e.g., m=1).

In some examples, the EMR data extractor 122 may determine a quality level associated with individual data instances of the EMR data 206. In examples, the EMR data extractor 122 may assign a quality level to an EMR data instance based on completeness of records, a level of detail of records, regularity of record updates, whether the records indicate a recurring physician and/or location of appointment (e.g., indicating a pattern of regular check-ups), and the like. For example, a first EMR data instance that indicates regular updates (e.g., yearly or more frequent), includes detailed diagnoses, includes recurring test results, and/or indicates regular doctor's appointments may be assigned a higher quality level than a second EMR data instance that indicates a few, unevenly-spaced doctor's appointments, few or no test results, and/or primarily emergency room or urgent care visits.

The training dataset 202 may be accessed by a training component 216 to train one or more AI/ML models 218 of the AI recommender system 124. In some examples, the training component 216 may be implemented on the remote computing device 118 and access the training data 202 over a network, such as the network 116. Alternatively, the training component 216 may be implemented on a computing device different from the remote computing device 118.

The training dataset 202 may be used to train feature detectors for features that are correlated with particular diseases, as described in U.S. patent application Ser. No. 17/709,950, filed Mar. 31, 2022, titled “Automated disease identification based on ophthalmic images,” which is incorporated by reference herein, as noted above. For example, U.S. patent application Ser. No. 17/709,950, provides Tables 1-5 correlating disease conditions with features related to an eye of a patient and/or the patient's EMR data. In some examples, the AI recommender system 124 may identify a set of diseases and train detectors for detecting known features associated with the set of diseases (e.g., as established by medical studies). However, there may be other correlations between the features 208, 212 and the diagnosis 214 that are not known e.g., medical studies have not been conducted to establish such correlations. As examples, some diagnoses may be correlated with features detected in the retinal images of the patient, a geographic region of the patient, and/or the patient's medical history. In addition, some correlations may be predictive in nature e.g., a subset of the features 208, 212, may be associated with a disease diagnosis in the future (e.g., a year later, 3 years later, 5 years later, etc.).

In examples of the present disclosure, the training component 216 may implement data mining techniques to determine correlations between the diagnosis 214 and one or more features of the eye features 208 and/or the EMR features 212. As a non-limiting example, the training component 216 may include frequent pattern mining (e.g., frequent itemset mining), and output candidate association rules, which may be single of multi-dimensional, that indicate that a set of features are associated with a particular diagnosis in the training dataset 202. The training component 216 may select candidate association rules that satisfy a minimum support threshold where the minimum support threshold indicates a minimum number of data instances in the training dataset 202 that support the association between the set of features and the particular diagnosis. The training component 216 may further analyze such candidate association rules by using statistical methods to determine a correlation value (e.g., Pearson's correlation coefficient, Spearman's coefficient, Cramer's coefficient, Kendall's coefficient, etc.) between the particular diagnosis and the set of features, and filter out candidate association rules where the respective correlation value is less than a minimum correlation threshold. In examples, the AI recommender system 124 may use the association rules that satisfy the minimum correlation threshold to generate a recommendation of screening for disease(s) or disease risk(s) indicated in the diagnosis. The AI recommender system 124 may also determine a confidence score based on the correlation value of the respective association rule. As a simplified example, an association rule that satisfies the minimum correlation threshold may indicate that a feature set (“optic disc edema,” “decreased AVR,” “high BMI,” “age>55”) correlates with a diagnosis of “obstructive sleep apnea.” In this example, if data associated with the patient 102 matches the feature set (“optic disc edema,” “decreased AVR,” “high BMI,” “age>55”), the AI recommender system 124 may recommend a screening for “obstructive sleep apnea” for the patient 102. Further, if the correlation value of the association rule was 0.7, the AI recommender system 124 may output the recommendation for screening with a confidence score of 0.7. In examples, the AI recommender system 124 may output the recommendation only if the confidence score associated with the recommendation is higher than a minimum threshold.

As another example, the training component 216 may train a Bayesian belief network based on the training data 202. Such a Bayesian belief network is characterized by conditional probability tables allowing a calculation of a probability of a diagnosis given a feature set. For example, the AI recommender system 124 may recommend a screening for a disease if an output probability of the disease, as computed by the trained Bayesian belief network, is higher than a threshold probability. In yet another example, the training component 216 may determine rules (e.g., if-then) from the training data 202 using techniques such as sequential covering algorithm, creating a hierarchical decision tree which may be used by the AI recommender system 124 to determine if a disease condition is reached (as a leaf node) based on the features 208, 212 corresponding to a particular patient, such as the patient 102.

In some examples, the training component 216 may perform temporal data mining to on the training dataset 202 to identify trends in the features/image 208, 210, 212 associated with disease risk or future diagnosis of a disease. For example, the training component 216 may use the timestamps associated with the features/image 208, 210, 212 to represent a same feature (e.g., systolic blood pressure) as a time-series, and use data mining techniques for mining patterns in time-series data to generate a predictive model indicating disease risk. In some examples, the training component 216 may determine changes over time by comparing the standard images 210 generated from retinal images captured at different times. In some examples, the training component 216 may generate derivative features to indicate time-series information e.g., “increasing blood pressure” may be added as a feature to the EMR features 212 before determining association rules. As an example, the training component 216, using time-series analysis, may establish a correlation between an increasing AVR ratio over time with obstructive sleep apnea, add an “increasing AVR ratio” as an eye feature, and this feature may appear, in an association rule in conjunction with other features, for detecting obstructive sleep apnea. In such an example, the AI recommender system 124 may generate a recommendation of screening for obstructive sleep apnea if a trend of AVR ratio computed from retinal images of a patient over time shows an increasing pattern, in conjunction with the other features identified in the association rule.

In some examples, the training component 216 may train classifiers for identifying one or more diseases and/or disease risks based on the eye features 208, the EMR features 212, and/or the standard images 210, as described in further detail with reference to FIG. 3. In examples, the AI/ML models 218 may store association rules (including temporal association rules) discovered as described above, as well as the classifiers which may be implemented as trained neural networks. In some examples, the training component 216 may use a first portion of the training dataset 202 for training, and a second (e.g., remaining) portion of the training dataset 202 for validation of the trained AI/ML models 218.

Various systemic diseases (e.g., fibromyalgia, Lyme disease, kidney disease, stroke risk, etc.) require multiple diagnostic tests, test results over an extended period of time (e.g., few months or years), and/or symptoms over an extended period of time for arriving at a diagnosis. The AI recommender system 124 may provide a faster route to arriving at the diagnosis by recommending screening for diseases based on a data-driven approach to identifying correlations between features of a patient's retinal images and their health records, as obtained during their regular health screenings at a doctor's office, and particular diseases. As discussed, a doctor (e.g., a primary care physician) conducting the regular health screening may not be able to interpret retinal images and therefore, may not be able to incorporate the features of the patient's retinal images in diagnosing diseases or disease risks. In addition, the AI recommender system 124 may recommend screening by identifying disease risk(s) based on the temporal associations identified in the training dataset 202.

Some examples of associations between sets of features and diseases are summarized below in Table 1.

TABLE 1

| Disease/ | |
|---|---|
| Disease risk | Features |
| Anemia | Eye features: color of optic disc, brightness level of |
| | blood vessels |
| | EMR features: tiredness, dizziness |
| Stroke/heart | Eye features: average diameter of blood vessels, change |
| disease risk | in topology of blood vessels |
| | EMR features: age, smoking status, high blood pressure |
| | measurements, smoker |
| Obstructive | Eye features: optic disc edema, decreasing AVR ratio, |
| sleep apnea | retinal arteriolar caliber |
| | EMR features: age, high BMI, morning headache, |
| | snoring, daytime sleepiness, mood change |
| Alzheimer's | Eye features: reduced capillary density, change in blood |
| disease | vessels |
| | EMR features: age, brain perfusion value in MRI, |
| | sedentary lifestyle |
| Lyme | Eye features: optic nerve inflammation, retinitis, scleritis |
| disease | EMR features: weakness, cognitive impairment, lives in |
| | tick-prone area, sensitivity to light |
| Fibromyalgia | Eye features: retinal nerve fiber layer (RNFL) thinning |
| | EMR features: pain throughout the body (over time), |
| | complete blood count (CBC) values in blood test, |
| | erythrocyte sedimentation rate (ESR) value |
| Parkinson's | Eye features: retinal thinning |
| disease | EMR features: changes in motion perception, age, tremor, |
| | stiffness |
| Multiple | Eye features: RNFL thinning, abnormal total macular |
| sclerosis | volume |
| (MS) | EMR features: visual impairment, muscle weakness |
| Chronic | Eye features: width of reflective erythrocyte column, AVR |
| kidney | value, density of vessel network |
| disease | EMR features: age, sex, ethnicity, diabetes, hypertension |

FIG. 3 illustrates an example system 300 for generating a recommendation for a patient, where the recommendation is based on output(s) of classifier(s) 302. In examples, the classifier(s) 302 may comprise trained machine learning (ML) models configured to identify a presence of disease(s) or disease risk(s) when provided, as inputs, features of retinal image(s) and health records of the patient. As illustrated, the system 300 includes an embodiment of the AI/ML models 218 of the AI recommender system 124 comprising the classifier(s) 302. In examples of the present disclosure, the AI recommender system 124 may use the AI/ML models 218 described with reference to FIG. 2, the AI/ML models 218 described in FIG. 3, or a combination thereof.

As described above with reference to FIG. 1, the image analysis system 120 may receive and/or identify (e.g., from a computer memory) one or more retinal image(s) 108. The retinal image(s) 108 may depict at least one eye of a patient, such as the patient 102. For example, the retinal image(s) 108 may include at least one OCT image and/or slit lamp image of an eye of the patient and a retina and/or fundus of the eye of the patient. The image analysis system 120 may determine ophthalmic features, including structural and color features, of the retinal image(s) 108 as well as a standardized retinal image, as described with reference to FIG. 1. Example ophthalmic features are described in U.S. patent application Ser. No. 17/709,950, filed Mar. 31, 2022, titled “Automated disease identification based on ophthalmic images,” which is incorporated by reference herein, as noted above. Additionally, the EMR data 112 may be processed by the EMR data extractor 122 to extract an EMR feature vector of the patient, as also described with reference to FIG. 1.

In examples, the AI recommendation system 124 may provide at least a subset of features from the ophthalmic features determined by the image analysis system 120, and the EMR feature vector from the EMR data extractor component 122 as inputs to the classifier(s) 302. In some examples, the subset may be based on correlations discovered between the features and particular disease(s), as discussed with reference to FIG. 2. Alternatively, or in addition, the AI recommendation system 124 may provide the standardized retinal image from the image analysis system 120 as input to the classifier(s) 302.

In some examples, the classifier(s) 302 may comprise a set of ML models (e.g., each ML model trained to output a confidence level associated with a single disease). In other examples, the classifier(s) 302 may comprise a multi-class ML model trained to output an indication of one or more of a set of diseases. As examples, the classifier(s) 302 may comprise CNNs, transformer-based models, RNNs, etc. In some examples, the classifier(s) 302 may be based on a transformer architecture, and a portion of the inputs (e.g., locations of the ophthalmic features), after tokenization, may include position encoding indicating a relative position of the input token (e.g., with respect to the standardized retinal image). In examples, the disease(s) evaluated by the classifier(s) 302 may correspond to the diseases listed in Table 1.

In examples, the set of ML models or the multi-class ML model of the classifier(s) 302 may be trained, during a training phase, by the training component 216 using the training dataset 202 described with reference to FIG. 2. In examples, the training component may divide the M data instances of the training dataset 202 into a first portion (e.g., comprising 80% of the M data instances) for training the classifier(s) 302, and a second portion (e.g., remaining 20% of the M data instances) of the training dataset 202 for validation of the classifier(s) 302. For example, the second portion of the training dataset 202 may be used as test data to evaluate performance of the trained classifier(s) 302 to prevent overfitting and local minima problems that may sometimes be encountered after training ML models.

As discussed, in some examples, the training component 216 may train a set of ML models, each ML model trained to output a disease indicator of disease indicators 304(1)-304(N) (e.g., corresponding to a first disease, a second disease, . . . , and an Nth disease), where the disease indicator may comprise a confidence level associated with a presence of the disease. The training component 216 may train each such individual disease classifier(s) 302, for example a classifier for an nth disease, using a first subset of instances of the training dataset 202 that indicates a diagnosis of the nth disease. In some examples, the training component 216 may also use, as negative examples, a second subset of instances of the training dataset 202 that indicates a diagnosis other than of the nth disease.

In examples, the AI recommender system 124 may input the subset of features and/or the standardized retinal image to each ML model of the set of ML models, and determine a confidence score or probability of each disease 1, . . . , N as an output of each of the set of ML models. In other examples, the AI recommender system 124 may input the subset of features to a multi-class classifier of the classifier(s) 302 and receive confidence scores or probability corresponding to the diseases 1, . . . , N as output. In either example, the AI recommender system 124 may adjust the confidence scores received by the classifier(s) 302 based on other factors. For example, the AI recommender system 124 may adjust the confidence scores based on the quality level of the EMR data 112 e.g., a confidence score may be adjusted lower based on a low quality level. In another example, the AI recommender system 124 may adjust the confidence scores based on a frequency or likelihood of a diagnosis e.g., confidence scores associated with a rare disease may be adjusted to be lower. In examples, the AI recommender system 124 may provide the confidence scores corresponding to each disease 1, . . . , N to an evaluator 306.

In examples, the evaluator 306 may compare the confidence scores to a minimum threshold, and provide a recommendation 308 for screening for disease(s) where the confidence score (e.g., the disease indicator 304(1)-(N)) associated with the disease(s) is higher than the minimum threshold. In some examples, the evaluator 306 may also take into account healthcare policies of a specific geographic location, as described with reference to FIG. 1, in determining the recommendation 308. The evaluator 306 may also determine a severity associated with the disease based on the confidence score (e.g., the disease indicator 304(1)-(N)) associated with the disease(s) being higher than a second threshold, higher than the minimum threshold. In some examples, the evaluator 306 may use separate threshold(s) and/or range(s) for different diseases or types of diseases and/or different categories of patients (e.g., categories based on age). In some examples, the evaluator 306 may generate the recommendation 308 to include textual summaries of the disease(s), severity associated with the disease(s), and/or a description of feature(s) used to determine the presence of the disease(s) e.g., by using generative AI techniques such as large-language models (LLMs).

In some examples, the system 300 may include receiving result(s) of follow-up screening 310 based on the recommendation 308. In examples, if the result(s) 310 indicates a presence of the disease(s) indicated for screening in the recommendation 308, the training component 216 may add the result(s) 310 as ground truth in the training dataset 202. Alternatively, if the result(s) 310 indicates that the disease(s) in the recommendation 308 were not present, the training component 216 may update the training dataset 202 to add the outputs of the image analysis system 120 and the EMR data extractor 122 as a new data instance in the training dataset 202, with an indication that the disease(s) screened for in the result(s) 310 were not present. The training component 216 may retrain the classifier(s) 302 and/or the mine for association rules, as described with reference to FIG. 2, periodically based on the updated training dataset.

As discussed herein, FIGS. 2 and 3 illustrates systems for training and using an AI recommender for providing recommendation(s) for screening a patient for disease(s) or disease risk(s) based on their retinal image(s) and EMR data. Other examples of training and using the AI recommender tailored for detecting specific disease conditions and abnormalities are also envisioned. For example, if medical studies become available showing a correlation between specific features of retinal image(s) and/or EMR data with particular disease outcomes, classifiers, such as the classifier(s) 302 may be trained to output a positive or negative indication of the particular disease outcomes when provided with the specific features as input.

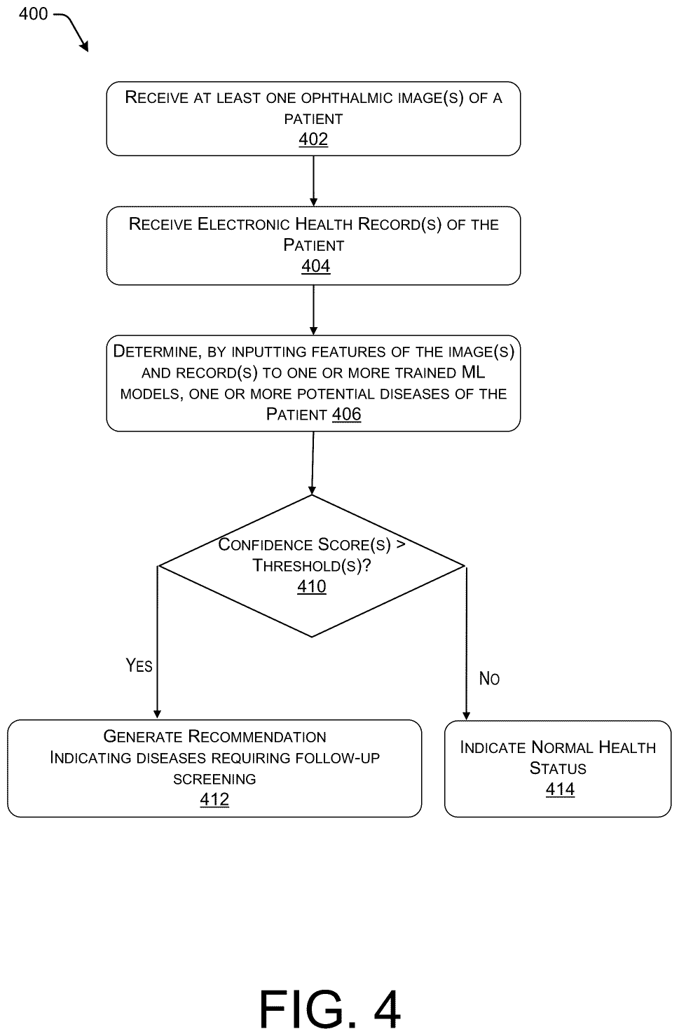

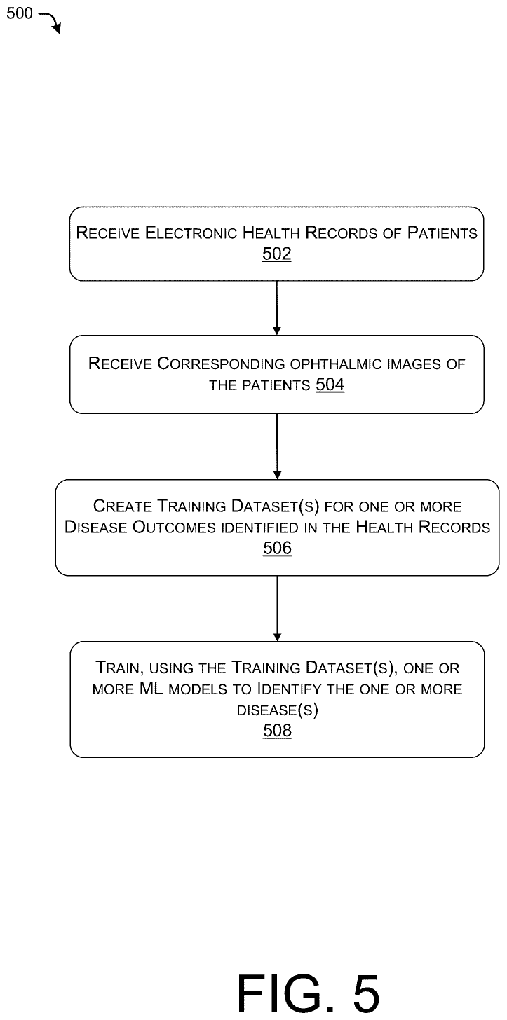

FIGS. 4 and 5 provide flow diagrams illustrating example methods for generating a recommendation for screening of disease(s), as described herein. The methods in FIGS. 4 and 5 are illustrated as collections of blocks in a logical flow graph, which represents sequences of operations that can be implemented in hardware, software, or a combination thereof. In the context of software, the blocks represent computer-executable instructions stored on one or more computer-readable storage media that, when executed by processor(s), perform the recited operations. Generally, computer-executable instructions include routines, programs, objects, components, data structures, and the like that perform particular functions or implement particular abstract data types. The order in which the operations are described is not intended to be construed as a limitation, and any number of the described blocks can be combined in any order and/or in parallel to implement the methods illustrated in FIGS. 4 and 5. In some embodiments, one or more blocks of the methods illustrated in FIGS. 4 and 5 can be omitted entirely.

The operations described below with respect to the methods illustrated in FIGS. 4 and 5 can be performed by any of the devices/systems 114, 118, 200, and 300 described herein, and/or by various components thereof. In particular, any of the operations described with respect to the methods illustrated in FIGS. 4 and 5 may be performed by the AI recommender system 124 or the training component 216. Unless otherwise specified, and for ease of description, the method illustrated in FIG. 4 will be described below with reference to the environment 100 shown in FIG. 1, and the method illustrated in FIG. 5 will be described below with reference to the systems 200, 300 shown in FIGS. 2 and 3.

With reference to an example process 400 illustrated in FIG. 4, at operation 402, the AI recommender system 124 may receive at least one ophthalmic image(s) of a patient, such as the patient 102. As described above with reference to FIG. 1, the ophthalmic image(s) may be captured by an optical imaging device 106 during a medical appointment (e.g., a routine health checkup) of the patient at a doctor's office. The ophthalmic image(s) may include one or more images 108 of a retina and/or a fundus of at least one eye. As non-limiting examples, the ophthalmic images may comprise OCT or OCTA images, fluorescence angiograms, CFP images, and the like.

At an operation 404, the AI recommender system 124 may include receiving electronic health record(s) of the patient. The electronic health record(s) (e.g., EMR data 112) may be accessed from an EMR system storing medical records of patients. The electronic health record(s) of the patient may include data indicating previous or current medical diagnoses of the patient, and a history of diagnostic tests, medications, or treatments received by the patient. In addition, the electronic health record(s) may indicate demographic information of the patient (e.g., age, sex, race, etc.), vital signs (e.g., blood pressure, blood oxygen level, heart rate, etc.), body mass index (BMI), lifestyle information (e.g., smoking or drug use status, physical activity level, diet, alcohol use, etc.), and the like, as measured during current and previous medical appointments.

At operation 406, the AI recommender system 124 may include determining, by inputting features of the ophthalmic image(s) and the electronic health record(s) to one or more trained ML models, one or more potential diseases or disease risk(s) of the patient. In examples, the features of the ophthalmic images and the electronic health records may be determined by the image analysis system 120 and the EMR data extractor 122, as described with reference to FIG. 1, and include the standardized retinal image. As described with reference to FIGS. 2 and 3, the ML models may be trained on large, anonymized training datasets containing retinal images, EMR data, and corresponding disease diagnoses of patients over an extended period of time. The one or more ML models (e.g., CNN, RNN, transformer-based models, etc.) may be trained to output a confidence score associated with a disease or disease risk of a set of diseases. In some examples, each ML model of the one or more ML models may be trained to detect a single disease. In some examples, the one or more ML models may comprise a decision tree, expert system, or a Bayesian belief network trained to compute a probability of a disease (e.g., corresponding to the confidence score) given the features as input. In examples, the one or more ML model(s) may output confidence score(s) associated with detection of the one or more potential diseases.

At operation 410, the AI recommender system 124 may compare the confidence score(s) obtained at the operation 406 to a minimum threshold. In examples, the minimum threshold may vary based on a type of disease, a characteristic of the patient (e.g., age), and/or health policies of a geographic region of the patient. For example, if the confidence score is higher than the minimum threshold (Operation 410—Yes), the AI recommender system 124 may generate, at an operation 412, a recommendation indicating disease(s) requiring follow-up screening. In examples, the recommendation may be provided to the patient and/or a healthcare provider caring for the patient, and may be added to the electronic health record(s) of the patient.

Alternatively, if the confidence score is not higher than the minimum threshold (Operation 410—No), the AI recommender system 124 may generate, at an operation 414, an indication of normal health status. In some examples, the indication of normal health status may not result in any action by the AI recommender system 124 and/or the healthcare provider receiving the indication.

FIG. 5 illustrates an example process 500 for training an AI recommender system, such as the AI recommender system 124, to identify one or more diseases or disease risk(s) based on ophthalmic images and electronic health records of a patient. The process 500 may be performed by a computing device that includes at least one processor and memory. In some examples, the process 500 may be performed by a computing device that is different from the computing device 118 implementing the AI recommender system 124, as described above with reference to FIG. 1.

At an operation 502, the training component 216 may receive electronic health records of a large number of patients. As described, the electronic health records (e.g., from the EMR system 110) may include data indicating previous or current medical diagnoses of the patients, and a history of diagnostic tests, medications, or treatments received by the patients. In addition, the electronic health record(s) may indicate demographic information of the patient, vital signs, lifestyle information, and the like, as measured over an extended period of time and/or during multiple medical appointments. The electronic health record(s) may also include physician's notes from the medical appointments, results of diagnostic tests performed on the patients, and treatment outcomes.

At an operation 504, the training component 216 may receive corresponding ophthalmic images of the patients. As examples, the images may include one or more of OCT images, slit lamp images, fundus images, or retinal images captured by one or more medical imaging devices configured to obtain the ophthalmic images. In some examples, the ophthalmic images may also be stored in the EMR system in association with patients' electronic health records, and may include images captured over an extended period of time. In examples, the images may include images illustrating disease conditions of a patient as well as images illustrating normal (e.g., disease-free) conditions. In examples, the electronic health records and the ophthalmic images of an individual patient may be identified as belonging to the same individual.

At an operation 506, the training component 216 includes creating a training dataset for one or more disease outcomes identified in the health records. As described with reference to FIG. 2, the training dataset may include features generated from the ophthalmic images and electronic health records e.g., as determined by the image analysis system 120 and the EMR data extractor 122. Each data instance of the training dataset may correspond to an individual patient, and include the features, including standardized retinal images, of the individual and diagnoses indicating the one or more disease outcomes (e.g., as obtained from the electronic health record corresponding to the individual). In some examples, the training dataset(s) may also include manual entries associated with one or more data instances, as provided by experts, indicating features (e.g., in the ophthalmic images) or diagnoses.

At an operation 508, the training component 216 may include training, using the training dataset created at the operation 506, one or more ML models to identify the one or more disease outcomes. For example, each training data instance may include a set of features as inputs, and a disease outcome (e.g., as indicated in the corresponding electronic health record) as a target output. In some examples, the training data instances for a particular disease may include a subset of the set of features as inputs, where the subset is based on association rules or correlations determined between the subset and the particular disease, as described with reference to FIG. 2. In some examples, the one or more ML models may include one or more NNs (e.g., CNNs, RNNs, graph neural networks, etc.), and the training component 216 may train the ML models based on optimizing parameter(s) of the models using techniques such as backpropagation. In some examples, the ML models may be decision trees, expert systems, or Bayesian belief networks, which may be trained by computing conditional probabilities of a disease outcome given the set of features.

As discussed, the ML models trained by the training component 216 may be used by the AI recommender system 124 to generate a recommendation for screening of a patient for diseases. In examples, the process 500 may be repeated periodically e.g., in response to receipt of additional data and/or passage of time over a time threshold, to keep the ML models updated based on current data.

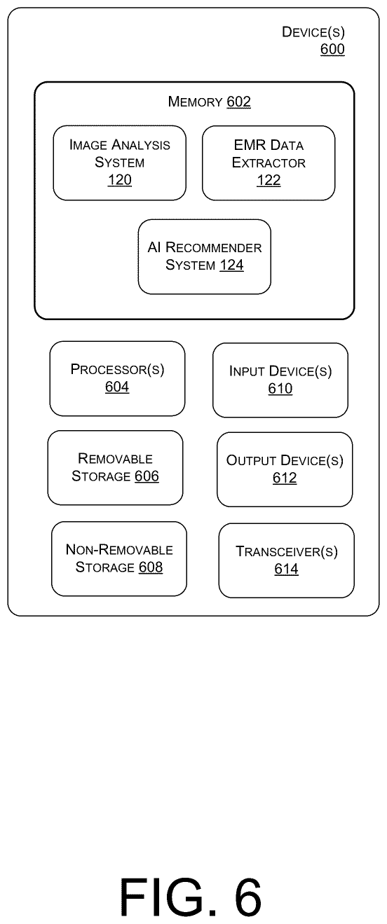

FIG. 6 illustrates at least one example device(s) 600 configured to enable and/or perform the some or all of the functionality discussed herein. Further, the device(s) 600 can be implemented as one or more server computers, a network element on a dedicated hardware, as a software instance running on a dedicated hardware, or as a virtualized function instantiated on an appropriate platform, such as a cloud infrastructure, and the like. It is to be understood in the context of this disclosure that the device(s) 600 can be implemented as a single device or as a plurality of devices with components and data distributed among them.

As illustrated, the device(s) 600, which may correspond to the computing device 118, may comprise a memory 602. The memory 602 can be used to store any number of functional components that are executable by the processor(s) 604. In examples, these functional components comprise instructions or programs that are executable by the processor(s) 604 and that, when executed, specifically configure the one or more processor(s) 604 to perform actions associated with providing a recommendation of screening for one or more the diseases. For example, the memory 602 may store one or more functional components, such as the image analysis system 120, the EMR data extractor 122, and the AI recommender system 124, as illustrated in FIG. 1. The memory 602 may also include files and databases used by the one or more functional components. The memory 602 may include volatile and nonvolatile memory and/or removable and non-removable media implemented in any type of technology for storage of information, such as computer-readable instructions, data structures, program modules, or other data.

As described herein, the processor(s) 604, can be a single processing unit or a number of processing units, and can include single or multiple processing cores, comprising a Central Processing Unit (CPU), a Graphics Processing Unit (GPU), or both CPU and GPU, or other processing unit known in the art. For example, the processor(s) 604 can be one or more hardware processors and/or logic circuits of any suitable type specifically programmed or configured to execute the algorithms and processes described herein. The processor(s) 604 can be configured to fetch and execute computer-readable instructions stored in the memory 602, which can program the processor(s) 604 to perform the functions described herein.

The device(s) 600 can also include additional data storage devices (removable and/or non-removable) such as, for example, magnetic disks, optical disks, or tape. Such additional storage is illustrated in FIG. 6 by removable storage 606 and non-removable storage 608. Tangible computer-readable media can include volatile and nonvolatile, removable and non-removable media implemented in any method or technology for storage of information, such as computer readable instructions, data structures, program modules, or other data. The memory 602, removable storage 606, and non-removable storage 608 are all examples of computer-readable storage media. Computer-readable storage media include, but are not limited to, RAM, ROM, EEPROM, flash memory or other memory technology, CD-ROM, Digital Versatile Discs (DVDs), Content-Addressable Memory (CAM), or other optical storage, magnetic cassettes, magnetic tape, magnetic disk storage or other magnetic storage devices, or any other medium which can be used to store the desired information and which can be accessed by the device(s) 600. Any such tangible computer-readable media can be part of the device(s) 600.

The device(s) 600 can also include input device(s) 610, such as a keypad, a cursor control, a touch-sensitive display, voice input device, etc., and output device(s) 612 such as a display, speakers, printers, etc. In some examples, the input device(s) 610 include a medical imaging device, such as the optical imaging device 106 described above with reference to FIG. 1. In particular implementations, a user can provide input to the device(s) 600 via a user interface associated with the input device(s) 610 and/or the output device(s) 612.

As illustrated in FIG. 6, the device(s) 600 can also include one or more wired or wireless transceiver(s) 614. For example, the transceiver(s) 614 can include a Network Interface Card (NIC), a network adapter, a LAN adapter, or a physical, virtual, or logical address to connect to the various base stations or networks (e.g., the network 116) contemplated herein, for example, or the various user devices and servers. To increase throughput when exchanging wireless data, the transceiver(s) 614 can utilize Multiple-Input/Multiple-Output (MIMO) technology. The transceiver(s) 614 can include any sort of wireless transceivers capable of engaging in wireless, Radio Frequency (RF) communication. The transceiver(s) 614 can also include other wireless modems, such as a modem for engaging in Wi-Fi, WiMAX, Bluetooth, or infrared communication.

Based at least on the description herein, it is understood that the AI recommender system and devices and methods of the present disclosure may be used to assist in identifying one or more vision diseases or disease risk(s), and recommending screening of the patient for the identified diseases. The AI recommender system may be trained on a large training dataset of anonymized patient data, and provide a recommendation to the patient based on retinal images and EMR data of the patient, as collected during a medical appointment at a doctor's office. The system described herein may also implement data mining techniques to discover associations between a set of features and a disease outcome based on the data in the training dataset. The recommendation may allow for screening of a patient for potential diseases for early diagnosis and treatment of diseases, before the diseases become more severe.

The foregoing is merely illustrative of the principles of this disclosure and various modifications can be made by those skilled in the art without departing from the scope of this disclosure. The examples described above are presented for purposes of illustration and not of limitation. The present disclosure also can take many forms other than those explicitly described herein. Accordingly, it is emphasized that this disclosure is not limited to the explicitly disclosed methods, systems, and apparatuses, but is intended to include variations to and modifications thereof, which are within the spirit of the following claims.

As a further example, variations of apparatus or process limitations (e.g., dimensions, configurations, components, process step order, etc.) can be made to further optimize the provided structures, devices and methods, as shown and described herein. In any event, the structures and devices, as well as the associated methods, described herein have many applications. Therefore, the disclosed subject matter should not be limited to any single example described herein, but rather should be construed in breadth and scope in accordance with the appended claims.

What is claimed is:

1. A method, comprising:
receiving, by a processor, an image of a retina of an eye of a patient;
receiving, by the processor and from an electronic medical record (EMR) of the patient, patient data corresponding to the patient;
determining, by the processor, a feature in the image;
determining, by the processor and by inputting the feature and at least a portion of the patient data as input to a machine learning (ML) model, a confidence level associated with a first disease;
determining, by the processor and based on the confidence level being higher than a threshold, a recommendation for screening of the patient based on the first disease; and
providing, by the processor and to an output device, an output indicating the recommendation.

2. The method of claim 1, wherein the feature comprises at least one of:
a brightness level of an optic disc of the retina,
a diameter of blood vessels of the retina,
a topology of the blood vessels of the retina,
an edema of the optic disc, or
an arteriovenous ratio (AVR).

3. The method of claim 1, wherein the patient data comprises at least one of:
an age of the patient,
a sex of the patient,
a race of the patient,
a smoking status of the patient,
a blood pressure measurement of the patient, or
one or more medical test results associated with the patient.

4. The method of claim 1, further comprising:
receiving, by the processor, follow-up information indicating whether the patient was diagnosed with the first disease;
augmenting, by the processor, a training dataset to include a data point comprising the follow-up information, the feature, and at least the portion of the patient data; and
updating, by the processor, the ML model by re-training with the augmented training dataset.

5. The method of claim 1, wherein the first disease comprises one of:
obstructive sleep apnea (OSA),
anemia,
heart disease,
kidney disease,
multiple sclerosis (MS), or
Alzheimer's disease.

6. The method of claim 1, wherein the ML model is trained, based on a training dataset, to identify, based on the image and the patient data as inputs, the confidence level associated with the first disease.

7. The method of claim 6, wherein the training dataset includes anonymized patient data and corresponding images of the retina associated with a plurality of patients, and an indication of normal health or one or more diseases associated with each respective patient.

8. The method of claim 7, wherein the anonymized patient data and the corresponding images of the retina are extracted from an electronic medical records (EMR) system.

9. The method of claim 1, wherein the ML model comprises an expert system indicating rules correlating the feature and the patient data with a probability of occurrence of the first disease.

10. A system, comprising:
memory;
a processor; and
computer-executable instructions stored in the memory and executable by the processor to perform operations comprising:
receiving an image of a retina of an eye of a patient;
receiving, from an electronic medical record (EMR) of the patient, patient data corresponding to the patient;
determining a feature in the image;
determining, by inputting the feature and at least a portion of the patient data as input to a machine learning (ML) model, a confidence level associated with a first disease;
determining, based on the confidence level being higher than a threshold, a recommendation for screening of the patient based on the first disease; and
providing, to the EMR of the patient, an output indicating the recommendation.

11. The system of claim 10, wherein the ML model is trained, based on a training dataset, to identify, based on the image and the patient data as inputs, the confidence level associated with the first disease.

12. The system of claim 11, wherein the training dataset includes anonymized patient data and corresponding images of the retina associated with a plurality of patients, and an indication of normal health or one or more diseases.

13. The system of claim 10, the operations further comprising:
receiving follow-up information indicating whether the patient was diagnosed with the first disease;
augmenting a training dataset to include a data point comprising the follow-up information, the feature, and at least the portion of the patient data; and
updating the ML model by re-training with the augmented training dataset.

14. The system of claim 10, wherein the ML model is based at least in part on determining, in a training dataset, a correlation between the first disease and the feature or the patient data.

15. The system of claim 10, wherein the first disease is one of: obstructive sleep apnea (OSA), anemia, heart disease, kidney disease, multiple sclerosis (MS), or Alzheimer's disease.

16. A non-transitory computer-readable storage medium storing processor-executable instructions that, when executed, cause one or more processors to:
receive, from an optical imaging device, an image of a retina of an eye of a patient;
access, from an electronic medical record (EMR) storage, EMR data of the patient;
determine a feature in the image;
determine, by inputting the feature and at least a portion of the EMR data as input to a machine learning (ML) model, a confidence level associated with a first disease; and
determine, based on the confidence level being higher than a threshold, a recommendation for screening of the patient based on the first disease.

17. The non-transitory computer-readable storage medium of claim of claim 16, wherein the ML model is trained based on a training dataset comprising anonymized patient data and corresponding images of the retina associated with a plurality of patients, and an indication of normal health or one or more diseases of respective patients.

18. The non-transitory computer-readable storage medium of claim of claim 16, wherein the ML model is based at least in part on determining, in a training dataset, a correlation between the first disease and the feature or the EMR data.

19. The non-transitory computer-readable storage medium of claim of claim 16, wherein:
the EMR data comprises at least one of: an age of the patient, a blood pressure measurement of the patient, or one or more medical test results associated with the patient, and
the feature comprises at least one of: a brightness level of an optic disc of the retina, a diameter of blood vessels of the retina, an edema of the optic disc, or an arteriovenous ratio (AVR).

20. The non-transitory computer-readable storage medium of claim of claim 16, wherein the first disease comprises one of: obstructive sleep apnea (OSA), anemia, heart disease, kidney disease, multiple sclerosis (MS), or Alzheimer's disease.
